(19) United States
(12) Patent Application Publication
(10) Pub. No.: US 2006/0069012 A1
(43) Pub. Date: Mar. 30, 2006

(54) Methods and compositions for treating plasticity in a subject

(76) Inventors: Anthony Joonkyoo Yun, Palo Alto, CA (US); Patrick Yuarn-Bor Lee, Menlo Park, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/238,108

(22) Filed: Sep. 27, 2005

Related U.S. Application Data
(60) Provisional application No. 60/614,679, filed on Sep. 29, 2004.

(51) Int. Cl. A61K 38/22; A61K 38/09; A61K 38/24

(52) U.S. Cl. 514/2

(57) ABSTRACT

Methods and compositions for at least maintaining plasticity in a system of a subject are provided. Embodiments of the subject methods include lowering long term potentiation in said subject in a manner effective to at least maintain plasticity of a system in said subject.

### CROSS-REFERENCE TO RELATED APPLICATIONS

Pursuant to 35 U.S.C. § 119 (e), this application claims priority to the filing date of U.S. Provisional Patent Application Ser. No. 60/614,679 filed Sep. 29, 2004; the disclosure of which application is herein incorporated by reference.

### BACKGROUND OF THE INVENTION

Plasticity, generally defined as the ability to maintain an undifferentiated state so that an organism can perform a variety of different tasks, plays an important role in a variety of biological systems. For example, cognitive plasticity plays an important role in the ability of an organism to learn complex tasks. However, the plasticity of various systems declines as organisms age.

### SUMMARY OF THE INVENTION

Methods and compositions for at least maintaining plasticity in a system of a subject are provided. Embodiments of the subject methods include lowering long term potentiation in said subject in a manner effective to at least maintain plasticity of a system in said subject.

The subject invention may be used to increase plasticity in a variety of different systems of a subject, including, but not limited to, a neuronal system, an immune system, a musculoskeletal system, a wound healing system, and the like.

### BRIEF DESCRIPTIONS OF THE DRAWINGS

FIG. 1 shows an exemplary embodiment of an electric energy applying device operatively positioned in a subject's body in accordance with embodiments of the subject methods.

### DEFINITIONS

“Plasticity” is a measure of an organism's ability to respond to an environmental stimulus in an adaptive manner. By adaptive is meant un-predetermined. As such, an organism exhibits “plasticity” if it responds to an environmental stimulus in an adaptive manner, such that it processes the nature of the stimulus, and responds thereto in a manner that has not been determined prior to the stimulus. Conversely, an organism exhibits little or no plasticity if it responds to a stimulus in a predetermined manner.

“Cognitive plasticity” refers to the ability to learn, e.g., the ability to learn complex tasks and concepts, analogous to the ability to learn of an organism that is undifferentiated such as a newborn or juvenile, e.g., a human from the time of birth to pre-pubertal age of about 10 years.

“Cartilage plasticity” refers to the ability to regenerate hyaline cartilage, analogous to the ability to regenerate hyaline cartilage in an organism that is undifferentiated such as a newborn or juvenile, e.g., a human from the time of birth to pre-pubertal age of about 10 years.

“Wound plasticity” refers to the ability to heal a wound, analogous to the ability to heal a wound in an organism that is undifferentiated such as a newborn or juvenile, e.g., a human from the time of birth to pre-pubertal age of about 10 years. Wound plasticity includes “scarring plasticity” which refers to the ability to minimize or prevent scarring.

“Allergy plasticity” refers to the ability to minimize or prevent allergic responses, analogous to the ability to minimize or prevent allergic responses in an organism that is undifferentiated such as a newborn or juvenile, e.g., a human from the time of birth to pre-pubertal age of about 10 years.

Long-term potentiation (LTP) may be characterized as a strengthening (or potentiation) of the connection between two nerve cells which lasts for an extended period of time (e.g., minutes to hours in vitro and hours to days and months or more in vivo). LTP may be induced by applying a sequence of short, high-frequency stimulations to an afferent (input) or efferent (output) pathway.

By “adjuvants” is meant a compound that, when used in combination with the one or more pharmacological agent compounds and/or compositions, augments or otherwise alters or modifies the resultant pharmacological and/or physiological responses.

### DETAILED DESCRIPTION OF THE INVENTION

Methods and compositions for at least maintaining plasticity in a system of a subject are provided. Embodiments of the subject methods include lowering long term potentiation in said subject in a manner effective to at least maintain plasticity of a system in said subject.

The subject invention may be used to at least maintain plasticity in a variety of different systems, including, but not limited to, a neuronal system, an immune system, a musculoskeletal system, a wound healing system, and the like.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All patents, patent applications and publications mentioned herein are incorporated herein by reference to disclose and describe the methods and/or materials in connection with which the patents, patent applications and publications are cited. The citation of any patent, patent application and publication is for its disclosure prior to the filing date and should not be construed as an admission that the present invention is not entitled to antedate such patent, patent application and publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

The figures shown herein are not necessarily drawn to scale, with some components and features being exaggerated for clarity.

As noted above, the subject methods are methods for at least maintaining plasticity in a system of a subject, where embodiments include at least maintaining plasticity in a system of a subject by lowering the long term potentiation (LTP) in the subject in a manner effective to at least maintain plasticity of a system in the subject. Embodiments include increasing plasticity in a system of a subject by lowering the LTP in the subject in a manner effective to increase plasticity of a system in the subject. Embodiments include lowering LTP using pharmacological agents and/or by applying electric energy to the subject in a manner effective to increase plasticity of a system of the subject. Although the subject invention is described below primarily with respect to increasing plasticity in a system of a subject by lowering the LTP in the subject in a manner effective to increase plasticity of a system in the subject, such description is for exemplary purposes only and is in no way intended to limit the scope of the invention. It is to be understood that the subject methods may be employed to, e.g., at least maintain plasticity in a system of a subject.

Embodiments include lowering LTP in a subject by administering an effective amount of an LTP lowering pharmacological agent to the subject. Pharmacological agents that may be administered to a subject in accordance with the subject invention include hormones, including hormone analogues and hormone precursors, such as melatonin and analogues or precursors thereof, and other neuropeptides described herein.

Embodiments include lowering LTP in a subject by modulating a receptor of a hormone, including receptors of hormone analogues and receptors of hormone precursors, such as melatonin receptors, receptors of melatonin analogues and receptors of melatonin precursors, and other receptors described herein. Receptors may be modulated using pharmacological and/or electrical means.

Embodiments also include lowering LTP in a subject by modulating a biological pathway of a hormone or neuropeptide.

The subject invention may be employed to at least maintain, e.g., increase, plasticity in a variety of systems in a subject. In certain embodiments, the systems are those that demonstrate a decrease in plasticity as the subject ages, e.g., after about the puberty phase of the subject. Exemplary systems include, but are not limited to, a neuronal system, an immune system, a musculoskeletal system, a wound healing system, and the like. As such, a variety of different types of plasticities may be at least maintained, e.g., increased, according to the subject invention. For example, with respect to at least maintaining, e.g., increasing, plasticity in a neuronal system of a subject, cognitive plasticity may be at least maintained, e.g., increased; with respect to at least maintaining, e.g., increasing, plasticity in a subject immune system, allergy plasticity may be at least maintained, e.g., increased; with respect to at least maintaining, e.g., increasing, plasticity in a musculoskeletal system of a subject, cartilage plasticity may be at least maintained, e.g., increased; with respect to at least maintaining, e.g., increasing, plasticity in a wound healing system of a subject, scarring plasticity may be at least maintained, e.g., increased.

Accordingly, a feature of embodiments of the subject methods is that plasticity in a system of a subject is at least maintained, e.g., increased, by lowering LTP of the subject. For example, plasticity in a system may at least be maintained at a certain level, where in certain embodiments plasticity in a system may be increased. For example, embodiments of the subject methods includes determining the plasticity in a system of a subject and then lowering LTP in the subject in a manner to at least maintain the plasticity at the level determined prior to lowering LTP. Embodiments include determining the plasticity in a system of a subject and then lowering LTP in the subject in a manner to increase the plasticity to a level greater than the level determined prior to lowering LTP.

The increase in plasticity is relative to the plasticity in the system of the subject absent the subject invention, where the increase may be about 1% greater or more as compared with the plasticity in the system of the subject absent the subject invention, e.g., about 5% greater or more in certain embodiments. Plasticity in certain systems may decrease as a subject ages. As such, in certain embodiments plasticity in a system of a subject may be increased to provide plasticity commensurate with the plasticity observed at a particular age or age range, which particular age or age range may depend on the type of subject. Plasticity may be increased in a system of a subject to be commensurate with average plasticity in a system of a like-subject of an age that is recognized as having high plasticity, e.g., a juvenile age. For example, plasticity in a system of a human subject may be increased to be commensurate with the average plasticity in the system of healthy human subjects of an age ranging from birth to pre-pubertal age of about 10 years. Likewise, different subjects such as other species may have different targeted plasticities, e.g., plasticity in a system of an avian subject may be increased to be commensurate with the average plasticity in the system of healthy avian juvenile subjects, e.g., for zebra finchs younger than about 40 days.

LTP may be lowered any suitable amount, where the amount may vary depending on the particular system/plasticity of interest. In certain embodiments, LTP may be lowered about 0.01% or more, e.g., from about 0.1 or more, e.g., from about 0.1% or more, e.g., from about 1% or more, relative to LTP in the subject absent the subject invention. In certain embodiments, LTP may be lowered to provide LTP commensurate with LTP observed at a particular age or age range of like-subjects, which particular age or age range may depend on the type of subject. LTP may be lowered to be commensurate with average LTP in a like-subject of an age that is recognized as having low LTP, e.g., a juvenile age. For example, for a human subject, LTP may be lowered to be commensurate with the average LTP in healthy human subjects of an age ranging from birth to pre-pubertal age of about 10 years. Likewise, other subjects may have different targeted LTP.

At least maintaining plasticity in a system of a subject, such as increasing plasticity in a system of a subject, by lowering LTP may be accomplished in any suitable manner, where embodiments include lowering LTP by administering an effective amount of a pharmacological agent to a subject and/or applying electrical energy to a subject. Embodiments include modulating receptors and biological pathways of a subject to lower LTP in a manner effective to increase plasticity, where embodiments include pharmacological and electrical energy means. Of interest are embodiments that lower LTP by increasing melatonin in a subject.

Administering a Pharmacological Agent to at Least Maintain Plasticity

In certain embodiments, the subject invention includes administering an effective amount of a pharmacological agent to a subject to at least maintain, e.g., increase, plasticity in a system of the subject. Any suitable pharmacological agents may be administered that are capable of lowering LTP in a manner effective to at least maintain, e.g., increase, plasticity and include, but are not limited to, hormones, hormone analogues and hormone precursors, and neuropeptides, such as melatonin, melatonin analogues and melatonin precursors, and other hormones and neuropeptides. For example, certain embodiments include administering an effective amount of a melatonin precursor such as serotonin (including serotonin analogues), tryptophan (including tryptophan analogues), and the like, to a subject to lower LTP. Other agents that may be administered include bombesin antagonists, galanin antagonists, gastrin agonists, melatonin agonists, melatonin antagonists, melanocorticotropin agonists, melanocorticotropin antagonists, melanocyte-stimulating hormone agonists, melanocyte-stimulating hormone antagonists, gastrin antagonists, ghrelin agonists, ghrelin antagonists, gonadotropin releasing hormone agonists, gonadotropin releasing hormone antagonists, follicle stimulating hormone agonist, follicle stimulating hormone antagonists, leutinizing hormone agonists, leutinizing hormone antagonists, growth hormone agonists, growth hormone antagonists, somatomedin agonists, somatomedin antagonists, oxytocin agonists, oxytocin antagonists, progesterone agonists, progesterone antagonists, relaxin agonists, serotonin agonists, serotonin antagonists, vasopressin agonists, vasopressin antagonists.

That is, embodiments of the subject methods include administering an effective amount, i.e., a therapeutically effective amount, of one or more pharmacological agents to a subject. By “effective amount” is meant a dosage sufficient to lower LTP a sufficient amount to increase plasticity, e.g., cognitive plasticity. The effective amount will vary with the age and physical condition of the subject, severity of the condition being treated, the duration of the treatment, the nature of any concurrent treatment, the pharmaceutically acceptable carrier used if any, and analogous factors within the knowledge and expertise of those skilled in the art.

In certain embodiments, more than one type of agent may be administered at the same or different times to treat the same or different condition. The effective amount of a given agent may vary somewhat from subject to subject, and may depend upon factors such as, but not limited to, the age and condition of the subject, the form of the agent, the route and method of delivery, etc., as noted above. Dosages may be determined in accordance with routine pharmacological procedures known to those skilled in the art.

Depending on the particular agent(s) administered to a subject, the agent(s) may be administered to a subject using any convenient means capable of resulting in the desired lowered LTP. Thus, a pharmacological agent may be incorporated into a variety of formulations for administration to a subject. A pharmacological agent may be formulated into pharmaceutical compositions by combination with appropriate, pharmaceutically acceptable carriers. By “pharmaceutically acceptable carrier” is meant a component such as a carrier, diluent, excipient, and the like of a composition that is compatible with the one or more pharmacological agents and other optional ingredients of the subject pharmacological agent compositions in that a pharmaceutically acceptable carrier may be combined with the pharmacological agent(s) without eliminating the biological or therapeutically effective activity of the one or more pharmacological agents, and is suitable for use in subjects as provided herein without undue adverse side effects (such as toxicity, irritation, allergic response, and death). Side effects are “undue” when their risk outweighs the benefit provided by the pharmaceutical agent. Non-limiting examples of pharmaceutically acceptable components include, but are not limited to, any of the standard pharmaceutical carriers such as phosphate buffered saline solutions, water, emulsions such as oil/water emulsions or water/oil emulsions, microemulsions, and various types of wetting agents. Accordingly, the pharmacological agents employed in the subject methods may be formulated into preparations in solid, semi-solid (e.g., gel), liquid or gaseous forms, such as tablets, capsules, powders, granules, ointments, solutions, suppositories, injections, inhalants and aerosols. As such, administration of a pharmacological agent may be achieved in various ways, including, but not limited to, oral, buccal (e.g. sub-lingual), rectal, topical (including both skin and mucosal surfaces, including airway surfaces), parenteral (e.g., subcutaneous, intramuscular, intradermal, intravenous and intrathecal), intraperiactivityal, transdermal, intracheal, intravaginal, endocervical, intrathecal, intranasal, intravesicular, in or on the eye, in the ear canal, etc., administration. In certain embodiments, one or more pharmacological agents may be administered via a transdermal patch or film system such as or analogous to that described, e.g., in U.S. Pat. Nos. 6,503,532; 5,302,395; 5,262,165; 5,248,501; 5,232,702; 5,230,896; 5,227,169; 5,212,199; 5,202,125; 5,173,302; 5,154,922; 5,139,786; 5,122,383; 5,023,252; 4,978,532; 5,324,521; 5,306,503; 5,302,395; 5,296,230; 5,286,491; 5,252,334; 5,248,501; 5,230,896; 5,227,169; 5,212,199; 5,202,125; 5,173,302; 5,171,576; 5,139,786; 5,133,972; 5,122,383; 5,120,546; 5,118,509; 5,077,054; 5,066,494; 5,049,387; 5,028,435; 5,023,252; 5,000,956; 4,911,916; 4,898,734; 4,883,669; 4,882,377; 4,840,796; 4,818,540; 4,814,173; 4,806,341; 4,789,547; 4,786,277; 4,702,732; 4,690,683; 4,627,429; and 4,585,452, the disclosures of which are herein incorporated by reference.

Embodiments may include pharmacological agent formulations for oral administration that may be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc., suitable for ingestion by the patient. Pharmaceutical preparations for oral use may be obtained through combination of at least one pharmacological agent with a solid excipient, optionally grinding a resulting mixture, and processing the mixture of granules, after adding suitable additional compounds, if desired, to obtain tablets or dragee cores. Suitable solid excipients include, but are not limited to, carbohydrate or protein fillers and include, but are not limited to sugars, including lactose, sucrose, mannitol, or sorbitol; starch from corn, wheat, rice, potato, or other plants; cellulose such as methyl cellulose, hydroxypropylmethyl-cellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate; with optional lubricants, such as talc or magnesium stearate; and if desired, with diluents, buffering agents, moistening agents, preservatives and flavoring agents.

Accordingly, pharmacological agent formulations suitable for oral administration in accordance with the subject invention may be present in discrete units, such as capsules, cachets, lozenges, tablets, and the like, each containing a predetermined amount of the active pharmacological agent; as a powder or granules; as a solution or a suspension in an aqueous or non-aqueous liquid; or as an oil-in-water or water-in-oil emulsion. Such pharmacological agent formulations may be prepared by any suitable method of pharmacy which includes, but is not limited to, bringing into association the active pharmacological agent and a suitable carrier (which may contain one or more optional ingredients as noted above). For example, pharmacological agent formulations for use with the subject invention may be prepared by uniformly and intimately admixing the active pharmacological agent(s) with a liquid or finely divided solid carrier, or both, and then, if necessary, shaping the resulting mixture. For example, a tablet may be prepared by compressing or molding a powder or granules containing the active pharmacological agent, optionally with one or more accessory ingredients. Compressed tablets may be prepared by compressing, in a suitable machine, the pharmacological agent in a free-flowing form, such as a powder or granules optionally mixed with a binder, lubricant, inert diluent, and/or surface active/dispersing agent(s). Molded tablets may be made by molding, in a suitable machine, the powdered pharmacological agent moistened with an inert liquid binder.

Pharmacological agents may also be administered in the form of suppositories for rectal administration of the drug. These formulations may be prepared by mixing a pharmacological agent with a suitable non-irritating vehicle or excipient which is solid at ordinary temperatures but liquid at the rectal temperatures and will therefore melt in the rectum to release the drug. Such materials include, but are not limited to, cocoa butter, carbowaxes and polyethylene glycols. Embodiments include one or more pharmacological agent(s) made into suppositories by mixing with a variety of bases such as emulsifying bases or water-soluble bases.

Pharmacological agents may also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

For example, embodiments may also include at least one pharmacological agent in an aerosolized, atomized or nebulized vapor form, e.g., administrable via a metered dose device or nebulizer, and the like such that embodiments also include aerosolizing, vaporing or nebulizing one or more pharmacological agents for administration to a subject. Accordingly, the one or more pharmacological agent agents may be utilized in aerosol formulation or an analogous formulation to be administered via inhalation or analogous means. The one or more pharmacological agents employed in the practice of the present invention may be formulated into pressurized acceptable propellants such as dichlorodifluoromethane, propane, nitrogen and the like.

Pharmacological agents may be delivered transdermally, by a topical route, formulated as applicator sticks, solutions, suspensions, emulsions, gels, creams, ointments, pastes, jellies, paints, powders, and aerosols. For example, embodiments may include a pharmacological agent formulation in the form of a discrete patch or film or plaster or the like adapted to remain in intimate contact with the epidermis of the recipient for a period of time. For example, such transdermal patches may include a base or matrix layer, e.g., polymeric layer, in which one or more pharmacological agent(s) are retained. The base or matrix layer may be operatively associated with a support or backing. Pharmacological agent formulations suitable for transdermal administration may also be delivered by iontophoresis and may take the form of an optionally buffered aqueous solution of the pharmacological agent compound. Suitable formulations may include citrate or bis/tris buffer (pH 6) or ethanol/water and contain a suitable amount of active ingredient.

Pharmacological agents may be delivered as microspheres for slow release in the body. For example, microspheres may be administered via intradermal injection of drug containing microspheres, which slowly release subcutaneously (see Rao, J. Biomater Sci. Polym. Ed. 7:623-645, 1995); as biodegradable and injectable gel formulations (see, e.g., Gao Pharm. Res. 12:857-863, 190.95); or, as microspheres for oral administration (see, e.g., Eyles, J. Pharm. Pharmacol. 49:669-674, 1997). Both transdermal and intradermal routes afford constant delivery for weeks or months.

Pharmacological agents may be provided as a salt and may be formed with one or more acids, including but not limited to hydrochloric, sulfuric, acetic, lactic, tartaric, malic, succinic, etc. Salts tend to be more soluble in aqueous or other protonic solvents that are the corresponding free base forms. In other cases, a preparation may be a lyophilized powder that is combined with buffer prior to use.

Pharmacological agents may administered parenterally, such as intravenous (IV) administration, and may include a solution of the pharmacological agent dissolved in a pharmaceutically acceptable carrier. Among the acceptable vehicles and solvents that may be employed, include, but are not limited to, water and Ringer's solution, an isotonic sodium chloride, etc. In addition, sterile fixed oils may conventionally be employed as a solvent or suspending medium. For this purpose any bland fixed oil may be employed including synthetic mono- or diglycerides. In addition, fatty acids such as oleic acid can likewise be used in the preparation of injectables. Accordingly, a pharmacological agent may be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of pharmacological agent in these formulations may vary widely, and will be selected based on fluid volumes, viscosities, body weight, and the like, in accordance with the particular mode of administration selected and the patient's needs. For IV administration, the formulation may be a sterile injectable preparation, such as a sterile injectable aqueous or oleaginous suspension. This suspension may be formulated according to the known art using those suitable dispersing or wetting agents and suspending agents. The sterile injectable preparation may also be a sterile injectable solution or suspension in a nontoxic parenterally-acceptable diluent or solvent, such as a solution of 1,3-butanediol, and the like. Accordingly, pharmacological agent formulations suitable for parenteral administration may include sterile aqueous and non-aqueous injection solutions of one or more active pharmacological agent agents, which preparations may be isotonic with the blood of the intended recipient. These preparations may contain, buffers and solutes which render the formulation isotonic with the blood of the intended recipient. Aqueous and non-aqueous sterile suspensions may include suspending agents and thickening agents. The formulations may be presented in single- or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind described above.

Pharmacological agents may be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the pharmacological agent into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). Accordingly, embodiments may include one or more pharmacological agents administered as liposomal formulations of the pharmacological agents. Methods for preparing liposomal suspensions are known in the art and thus will not be described herein in great detail. Briefly, in those embodiments where the pharmacological agent is an aqueous-soluble pharmacological agent, the pharmacological agent may be incorporated into lipid vesicles using conventional liposome technology. In such instances, due to the water solubility of the pharmacological agent, the pharmacological agent may be substantially entrained within the hydrophilic center or core of the liposomes. The lipid layer employed may be of any conventional composition and may either contain cholesterol or may be cholesterol-free. When the pharmacological agent of interest is water-insoluble, the pharmacological agent may be substantially entrained within the hydrophobic lipid bilayer which forms the structure of the liposome employing conventional liposome formation technology. In either instance, the liposomes which may be produced may be reduced in size, as through the use of standard sonication and homogenization techniques. Embodiments of liposomal formulations containing the pharmacological agent of interest may be lyophilized to produce a lyophilizate which may be reconstituted with a pharmaceutically acceptable carrier, such as water, to regenerate a liposomal suspension.

A pharmaceutical composition of the subject invention may optionally contain, in addition to a pharmacological agent, at least one other therapeutic agent useful in the treatment of a condition. Such other compounds may be of any class of drug or pharmaceutical agent, including but not limited to antibiotics, anti-parasitic agents, antifungal agents, anti-viral agents, anti-tumor agents, anti-neurodegenerative agents and anti-psychotic agents. When administered with anti-parasitic, anti-bacterial, anti-fungal, anti-tumor, anti-viral agents, anti-neurodegenerative, and anti-psychotic agents and the like, pharmacological agents may be administered by any method and route of administration suitable to the treatment of the condition, typically as pharmaceutical compositions.

Pharmacological agents may include pharmacological agent compositions that may be prepared from water-insoluble compounds, or salts thereof, such as aqueous base emulsions. In such embodiments, the pharmacological agent composition will typically contain a sufficient amount of pharmaceutically acceptable emulsifying agent to emulsify the desired amount of the pharmacological agent. Useful emulsifying agents include, but are not limited to, phosphatidyl cholines, lecithin, and the like.

As noted above, in addition to active pharmacological agent agents, the pharmaceutical pharmacological agent compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Furthermore, pharmacological agent compositions may, though not always, contain microbial preservatives. Microbial preservatives that may be employed include, but are not limited to, methylparaben, propylparaben, and benzyl alcohol. The microbial preservative may be employed when the pharmacological agent formulation is placed in a vial designed for multidose use. Pharmaceutical pharmacological agent compositions for use in practicing the subject methods may be lyophilized using techniques well known in the art.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Embodiments may also include administration of at least one pharmacological agent using a pharmacological delivery device such as, but not limited to, pumps (implantable or external devices), epidural injectors, syringes or other injection apparatus, catheter and/or reservoir operatively associated with a catheter, etc. For example, in certain embodiments a delivery device employed to deliver at least one pharmacological agent to a subject may be a pump, syringe, catheter or reservoir operably associated with a connecting device such as a catheter, tubing, or the like. Containers suitable for delivery of at least one pharmacological agent to a pharmacological agent administration device include instruments of containment that may be used to deliver, place, attach, and/or insert the at least one pharmacological agent into the delivery device for administration of the pharmacological agent to a subject and include, but are not limited to, vials, ampules, tubes, capsules, bottles, syringes and bags.

In certain embodiments, a pharmaceutically acceptable carrier may be preservative free. By “preservative free” is meant the substantial absence of chemical, antibacterial, antimicrobial, or antioxidative additives, or the like, from the pharmaceutically acceptable carriers of the present invention. “Substantial absence” may mean that no preservative is present in the compositions or that trace amounts may be present that impart no detectable effect otherwise attributable to a preservative. For example, the pharmaceutically acceptable carrier may be characterized by the substantial absence of chemical, antibacterial, antimicrobial, or antioxidative additives or the like (e.g., contain less than about 5.0, 4.0, 3.0, 2.0, 1.0, 0.5, 0.1, 0.05, 0.01, or even about 0.00 percent by weight of a preservative). Further, such formulations may be substantially or essentially free of alcohols such as ethanol (e.g., contain less than about 5.0, 4.0, 3.0, 2.0, 1.0, 0.5, 0.1, 0.05, 0.01, or even about 0.00 percent by weight of alcohols such as ethanol). Examples of suitable pharmacological agent formulations include, but are not limited to, formulations that include one or more active pharmacological agent agents and physiological saline solution (optionally including other typical ingredients such as other active agents and buffers).

As noted above, in pharmaceutical dosage forms, an agents may be administered alone or with or in appropriate association, as well as in combination, with other pharmaceutically active compounds. As used herein, “administered with” means that at least one pharmacological agent and at least one other adjuvant (including one or more other pharmacological agents) are administered at times sufficiently close that the results observed are indistinguishable from those achieved when one pharmacological agent and at least one other adjuvant (including one or more other pharmacological agents) are administered at the same point in time. The pharmacological agent and at least one other adjuvant may be administered simultaneously (i.e., concurrently) or sequentially. Simultaneous administration may be carried out by mixing the at least one pharmacological agent and at least one other adjuvant prior to administration, or by administering the pharmacological agent and at least one other adjuvant at the same point in time. Such administration may be at different anatomic sites or using different routes of administration. The phrases “concurrent administration,” “administration in combination,” “simultaneous administration” or “administered simultaneously” may also be used interchangeably and mean that the at least one pharmacological agent and at least one other adjuvant are administered at the same point in time or immediately following one another. In the latter case, the at least one pharmacological agent and at least one other adjuvant are administered at times sufficiently close that the results produced are synergistic and/or are indistinguishable from those achieved when the at least one pharmacological agent and at least one other adjuvant are administered at the same point in time. Alternatively, a pharmacological agent may be administered separately from the administration of an adjuvant, which may result in a synergistic effect or a separate effect. The methods and excipients described herein are merely exemplary and are in no way limiting.

Unit dosage forms for oral or rectal administration such as syrups, elixirs, and suspensions may be provided wherein each dosage unit, for example, teaspoonful, tablespoonful, tablet or suppository, contains a predetermined amount of a pharmacological agent. Similarly, unit dosage forms for injection or intravenous or other suitable administration route may include the pharmacological agent(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of pharmacological agent calculated in an amount sufficient to produce the desired effect in association with a pharmaceutically acceptable diluent, carrier or vehicle. The specifications for the unit dosage forms of pharmacological agents of the present invention depend on, for example, the particular pharmacological agent(s) employed and the effect to be achieved, the pharmacodynamics associated with the particular pharmacological agent(s) in the subject, etc.

Embodiments include administering an effective amount of a first agent and an effective amount of a second agent. For example, embodiments may include administering a first agent and a second agent to provide an enhanced therapeutic effect. By “enhanced therapeutic effect” is meant that at least the initial lowering of LTP occurs more quickly and/or is of greater magnitude with a combination of the pharmacological agents, as compared to the same doses of each component given alone; or that doses of one or all component(s) are below what would otherwise be a minimum effective dose (a “sub-MED”).

Ant two pharmacological agents may be given in close enough temporal proximity to allow their individual therapeutic effects to overlap. For example, embodiments of the subject invention include the co-timely administration of a first and second agent, where “co-timely” is meant administration of a second pharmacological agent to lower LTP while a first pharmacological agent is still present in a subject in a therapeutically effective amount. It is to be understood that in some instances this will require sequential administration. Alternatively, multiple routes of administration may be employed, e.g., intravenous or subcutaneous injection combined with oral administration, and the like.

Embodiments also include pharmaceutical compositions in unit dosage forms that are useful in lowering LTP which contain more than one type of pharmacological agent. In other words, a single agent administration entity may include two or more pharmacological agents. For example, a single tablet, capsule, dragee, trocheem suppository, syringe, and the like, combining two or more pharmacological agents would be a unit dosage form. The therapeutic agents present in a unit dosage form may be present in amounts such that, upon administration of one or more unit doses of the composition, a subject experiences, e.g., a longer lasting efficacy than with the administration of either agent alone and/or greater magnitude of LTP lowering and/or quicker lowering of LTP. Such compositions may be included as part of a therapeutic package in which one or more unit doses are placed in a finished pharmaceutical container. Labeling may be included to provide directions for using the composition for increasing plasticity by lowering LTP. The actual amounts of each agent in such single unit dosage forms may vary according to the specific compositions being utilized, the particular compositions formulated, the mode of application, the particular route of administration, and the like, where dosages for a given subject may be determined using conventional considerations, e.g., by customary comparison of the differential activities of the subject compositions and of a known agent, or by means of an appropriate, conventional pharmacological protocol.

Applying Electrical Energy to at Least Maintain Plasticity

As noted above, certain embodiments include employing electrical modulation, i.e., applying electrical energy, to lower LTP in a manner effective to at least maintain, e.g., increase, plasticity in a system of a subject, e.g., to modulate a receptor or biological pathway to lower LTP to at least maintain, e.g., increase, plasticity.

Any suitable area may be targeted for electrical modulation. Areas that may be targeted include, but are not limited to, pre- and post ganglionic nerve fibers, as well as ganglionic structures, efferent and afferent nerve fibers, synapses, etc., and combinations thereof in certain embodiments. In certain embodiments, activity in a given nerve fiber may be electrically modulated in more than one area of the nerve fiber. In certain embodiments, electrical energy is applied to modulate synaptic efficiency, e.g., to increase or decrease the sensitivity of a synapse and include modulating presynpatic neurons.

As such, areas which may be targeted with electrical energy include, but are not limited to, pre- and post ganglionic nerve fibers, ganglionic structures, efferent and afferent nerve fibers, the hypothalamus, receptors any receptor described herein, afferent autonomic nerves (sympathetic and parasympathetic). Embodiments include receptors of the hypothalamus, including hormonal receptors on the hypothalamus. In certain embodiments, a given nerve fiber or the like may be targeted for electrical modulation in more than one area of the nerve fiber. Targeted areas of the nervous system which may be targeted in accordance with the subject invention include, but are not limited to, vagus nerve, otic ganglion, and sphenopalatine ganglion, internal carotid nerve and plexus, middle and superior cervical sympathetic ganglion; vertebral ganglion; cervicothoracic ganglion; sympathetic trunk; cervical cardiac nerves; cardiac plexus; thoracic aortic plexus; celiac ganglion; celiac trunk and plexus; superior mesenteric ganglion; superior mesenteric artery and plexus; intermesenteric plexus; inferior mesenteric ganglion; inferior mesenteric artery and plexus; superior hypogastric plexus; hypogastric nerves; vesical plexus; thoracic cardiac nerves; sympathetic trunk; 6th thoracic sympathetic ganglion; gray and white rami communicantes; greater, lesser and least splanchnic nerves; aorticorenal ganglion; lumbar splanchnic nerves; gray rami communicantes and sacral splanchnic nerves; and the like, or a combination of two or more of the above.

A number of different devices may be employed in accordance with the subject invention to lower LTP. For example, device and systems for applying electrical energy to a subject and which may be adapted for use in the subject invention are described, e.g., in copending U.S. patent application Ser. Nos. 10/661,368, 10/871,366 and elsewhere, the disclosures of the U.S. patent applications are herein incorporated by reference. Such devices may be positioned directly on a targeted area, e.g., positioned below the skin of a subject directly on or adjacent a portion of the nervous system (e.g., one or more nerve fibers) such as an implantable device, or may be an external device (i.e., some or all of the device may be external to the subject). In accordance with the subject invention, one or more electrodes or electrical contacts may be positioned directly on or adjacent a targeted area, where the one or more electrodes may be surgically implanted, e.g., directly on or adjacent a targeted nerve fiber of a subject. In certain embodiments, an immunomodulator such as a steroid or the like, may be incorporated into a surface contacting area of a device, e.g., to minimize inflammation of the targeted site.

An electric energy applying device typically includes a stimulator such as an electrode, a controller or programmer and one or more connectors for connecting the stimulating device to the controller. In certain embodiments more than one electrode may be employed. In further describing representative electrodes, such are described in the singular, but it will be apparent that more than one electrode may be used, where such may be the same or may be different in one or more aspects. Accordingly, the description of an exemplary electrode suitable for use in the subject methods is applicable to other electrodes that may be employed.

The electrode employed in the subject invention is controllable to provide output signals that may be varied in voltage, frequency, pulse width, current and intensity. The electrode may be one that provides both positive and negative current flow from the electrode and/or may be capable of stopping current flow from the electrode and/or changing the direction of current flow from the electrode. For example, embodiments include an electrode that is controllable in these respects, i.e., controllable in regards to producing positive and negative current flow from the electrode, stop current flow from the electrode, change direction of current flow from the electrode, and the like. In certain embodiments, the electrode has the capacity for variable output, linear output and short pulse width.

The energy source for the electrical output may be provided by a battery or generator such as a pulse generator that is operatively connected to the electrode. The energy source may be positioned in any suitable location such as adjacent to the electrode (e.g., implanted adjacent the electrode), or a remote site in or on the subject's body or away from the subject's body in a remote location and the electrode may then be connected to the remotely positioned energy source using wires, e.g., may be implanted at a site remote from the electrode or positioned outside the subject's body in certain instances. Of interest are implantable generators analogous to a cardiac pacemaker.

The electrode may be mono-polar, bipolar or multi-polar. In order to minimize the risk of an immune response triggered by the subject against the device and minimize damage such as corrosion and the like to the device from other biological fluids, etc., the electrode and any wires and optional housing materials are made of inert materials such as for example silicon, metal, plastic and the like. For example, a multi-polar electrode having about four exposed contacts (e.g., cylindrical contacts may be employed).

A controller or programmer may also coupled with an electric energy applying device. The programmer is typically one or more microprocessors under the control of a suitable software program. Other components of the programmer will be apparent to those of skill in the art, e.g., analog to digital converter, etc.

The electric energy applying device may be pre-programmed for desired parameters. In certain embodiments the parameters are controllable such that the electrode signal may be remotely modulated to desired settings without removal of the electrode from its targeted position. Remote control may be performed, e.g., using conventional telemetry with an implanted electric signal generator and battery, an implanted radiofrequency receiver coupled to an external transmitter, and the like. In certain embodiments, some or all parameters of the electrode may be controllable by the subject, e.g., without supervision by a physician. For example, a magnetic signal may be employed. In such embodiments, one or more magnets may be employed such that upon bringing a magnet in proximity to or away from the power source such as a pulse generator, the magnet may be employed to interfere with the electronic circuitry thus modulating the power—either increasing or decreasing the power supplied depending on whether the magnet is brought in proximity or moved away from the power source.

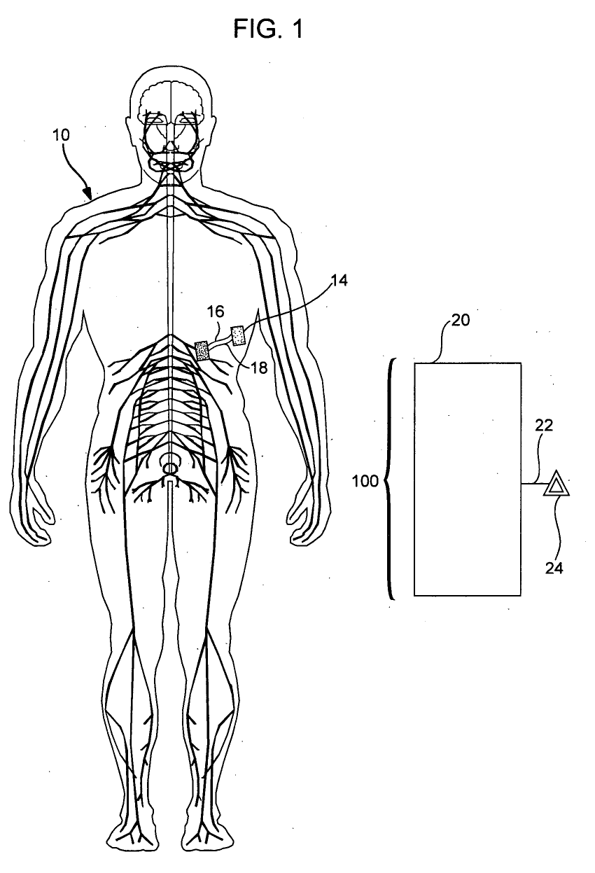

FIG. 1 shows an exemplary embodiment of an electric energy applying device 100. Device 100 may be implanted in a suitable position of a subject's body 10. One or more leads 23 are shown positioned to stimulatory or inhibitory electrical energy. Device 100 include energy source 14 which may take the form of a modified signal generator Model 7424 manufactured by Medtronic, Inc. under the trademark Intrel II. Lead 23 may take the form of any suitable lead, such as any of the leads that are sold with the Model 7427 and is coupled to energy source 14 by one or more conventional conductors 16 and 18. Lead 23 may include a paddle lead, a lead having one or more electrodes and/or catheters, or a combination catheter/lead capable of providing electrical impulses and pharmacological delivery. In certain embodiments, a lead may be composed of concentric tubes such as made of platinum or other like material. The tubes may be coated with a polymer except for the distal portions that may serve as the electrodes. Conductive wires carrying energy to the electrodes may be in the interior of the concentric tubes. Optionally, a distal electrode end may include a small recording microelectrode to help assist in the actual placement of the lead.

The present invention may be operated as an open-loop controlled system. In an open-loop system, the physician or patient may at any time manually or by the use of pumps or motorized elements adjust treatment parameters such as pulse amplitude, pulse width, pulse frequency, or duty cycle. Optionally, the present invention may incorporate a closed-loop control system which may automatically adjust the electrical parameters in response to a sensed parameter or condition of a subject. Under a closed-loop feedback system to provide automatic adjustment of parameters of the electrodes, a sensor that senses a condition of the body is utilized. More detailed descriptions of sensors that may be employed in the practice of the subject invention, and other examples of sensors and feedback control techniques that may be employed are disclosed in U.S. Pat. No. 5,716,377, which is incorporated herein by reference.

As shown in FIG. 1, the distal end of lead 23 terminates in one or more delivery elements such as stimulation electrodes which may be implanted using conventional surgical techniques. The type of treatment that is desired determines the location of the electrodes. Any number of electrodes may be used for various applications. Each of the electrodes may be individually connected to energy source 14 through lead 23 and conductors 16 and 18. Lead 23 may be surgically implanted either by a laminotomy or by a needle.

Energy source or signal generator 14 may be programmed to provide a predetermined stimulation (or inhibition) dosage in terms of pulse amplitude, pulse width, pulse frequency, or duty cycle. As shown, a programmer 20 may be utilized to provide stimulation (or inhibition) parameters to the delivery device via any suitable technology, e.g., using telemetry and the like. For example, in using telemetry, programmer 20 may be coupled to an antenna 24 via conductor 22. In certain embodiments, the programmer may be positioned, e.g., implanted, inside body 10. For example, in certain embodiments the programmer may be integrated with the energy source, electrode, etc., for example as a single unit.

Device 100 may optionally include one or more sensors to provide closed-loop feedback control of the treatment and/or electrode positioning. One or more sensors (not shown) may be attached to or implanted into a portion of a subject's body suitable for detecting a physical and/or chemical indicator of the subject. For example, sensing feedback may be accomplished, e.g., by a mechanical measure within a lead or an ultrasound or other sensor to provide information about the treatment parameters, lead positioning, LTP, etc.

Operative placement of a suitable electric energy applying device may be accomplished using any suitable technique. An electrode introducer needle may be employed to implant the electrode on or proximate to the area of interest. The size of the introducer needle may vary depending on the diameter of the electrode, etc., where in certain embodiments the electrode introducer needle may be a 12-gauge, 14-gauge, 16-gauge, 18-gauge, 20-gauge needle or 22-gauge needle, e.g., an electrode introducer needle available from Radionics in the Sluyter-Mehta kit as SMK 100 mm 2 mm active tip cannula. However, it should be understood that other electrode introducer needles may be used as appropriate to the needs and skill level of the practitioner performing the surgical procedure.

At least one imaging apparatus such as a CT scan, MRI apparatus, ultrasound apparatus, fluoroscope, or the like, may be employed to monitor the surgical. For exemplary purposes only, the subject method will be described using a fluoroscope, where such is in no way intended to limit the scope of the invention. The subject is placed in a suitable position for access e.g., supine, on a fluoroscopy table, with the patient's nose pointing vertically. The fluoroscope is then adjusted to a straight lateral position. And the entry point for the insertion of the electrode is determined.

Once the entry point is determined, the skin overlying the entry point is shaved and prepared with antiseptic solution. A 25-gauge needle may be used to inject a subcutaneous local anesthetic (such as, for example, 2 cc of 2% lidocaine) into the skin and subcutaneous tissues overlying the entry point. In addition to the local anesthetic, the patient may be given intravenous sedation and prophylactic antibiotics prior to commencement of the implantation procedure if desired.

The electrode introducer needle is inserted at the entry point and advanced. The fluoroscope may be adjusted as the needle is advanced. Once the needle is positioned the stylet is withdrawn from the electrode introducer needle. Once the implanted electrode is in place, the end of the electrode that is outside the skin is carefully held in place against the skin. The electrode introducer needle may then be slowly removed, leaving the implanted electrode in place. At this point, if desired, a few small subcutaneous sutures may be placed around the electrode to hold it in the desired position.

Once the needle has been completely removed and the implanted electrode is in the final position, then the proximal part of the electrode that is coming out of the skin may be secured to the skin of the subject, e.g., by adhesive tape. Additionally, a small incision may be made on the skin at the area the electrode exits the face. Then several subcutaneous sutures may be placed around the electrode to hold it in place. The distal end of the electrode may then be connected to an extension wire or catheter, which is tunneled to the subclavicular area, or another region which will house the device used as an energy source for the implanted electrode. The device or devices used to control the electrode may be surgically implanted in the desired region by procedures known in the art, such as have been applied in surgical neuromodulation therapies used to treat Parkinson's disease.

In certain embodiments of the subject invention, an electrode may be utilized which, instead of or in addition to delivering electric impulses to at least a portion of the autonomic nervous system, delivers a pharmacological agent to at least a portion of the autonomic nervous system. For example, an electrode may be used that has a small port at its tip which is connected to a reservoir or pump containing a pharmacological agent. The pharmacological agent delivery electrode may be implanted using an analogous procedure as that described above for the autonomic system modulating-electrode. In certain embodiments the reservoir or pump may also be implanted in the subject's body, analogous to that described above for the electrical impulse generator. The pharmacological agent delivery electrode may be controllable such that the amount of pharmacological agent delivered, the rate at which the pharmacological agent may be delivered, and the time period over which the pharmacological agent is delivered may be adjusted.

In embodiments in which electrical energy is used to increase plasticity, any suitable protocol may be used, where certain protocols include using an electric energy applying device to deliver a suitable amount of electrical energy to a subject. Once an electric energy applying device is positioned in a suitable position on or about one or more targeted areas electrical energy is applied thereto for a period of time sufficient to provide the desired lowered LTP (increase in plasticity). This period of time will vary depending on the area (e.g., the nerve fiber) being treated, the condition being treated, etc. Certain embodiments include simultaneously monitoring (i.e., in “real time”) the as aspect of the nervous system such that a given nerve fiber may be electrically stimulated (or electrically inhibited) until the desired result is observed. Still further, in many embodiments once the desired result is achieved, a targeted area may be repeatedly electrically stimulated (or inhibited) one or more times to maintain the desired state such that the subject methods may be repeated one or more times, i.e., the subject methods include chronically applying electrical energy to a subject, such as chronically applying electrical energy to one or more nerve fibers. For example, in certain embodiments electrical stimulation (e.g., intermittent mild electrical pulses) may be delivered to a given area of the nervous system, twenty-four hours a day for a period of days, weeks, months, or even years in certain embodiments.

During the period of time that electrical energy is applied to a given area, the electrical energy may be substantially continuous, including continuous or intermittent (i.e., pulsed or periodic), where in many embodiments the electrical energy is in the form of electrical pulses. In other words, in certain embodiments electrical energy may be given continuously during the above-described period of time and in certain embodiments electrical energy may be given to an area in a pulsed or intermittent manner during the period of time described above.

In accordance with the subject methods to apply electrical energy to a subject, once operatively positioned the electric energy applying device is activated to provide an electrical signal to the targeted area in a manner lower LTP in a manner effective to increase plasticity in a system of the subject.

In practicing the subject methods, activation of the electric energy applying device directly applies the electrical output of the device, i.e., electrical impulses, to the targeted area. The exact parameters of the applied electrical energy may vary depending on the particular subject, condition being treated, etc. For example, an electronic current wave may be provided when the electrical energy is applied. In certain embodiments, the current wave includes current waves of high frequency, e.g., high frequency pulses, where the current wave may also include low frequency amplitude modulation. In certain embodiments, a plurality of high frequency bursts of current pulses may be applied in addition to the application of underlying low frequency continuous stimulus. Stimulation may be monopolar or multipolar.

For example, to stimulate a targeted area, voltage or intensity may range from about 1 millivolt to about 1 volt or more, e.g., 0.1 volt to about 50 volts, e.g., from about 0.2 volt to about 20 volts and the frequency may range from about 1 Hz to about 2500 Hz, e.g., about 1 Hz to about 1000 Hz, e.g., from about 2 Hz to about 100 Hz in certain embodiments. In certain embodiments a pure d-c voltages may be employed. The pulse width may range from about 1 microsecond to about 2000 microseconds or more, e.g., from about 10 microseconds to about 2000 microseconds, e.g., from about 15 microseconds to about 1000 microseconds, e.g., from about 25 microseconds to about 1000 microseconds. The electrical output may be applied for at least about 1 millisecond or more, e.g., about 1 second, e.g., about several seconds, where in certain embodiments the stimulation may be applied for as long as about 1 minute or more, e.g., about several minutes or more, e.g., about 30 minutes or more may be used in certain embodiments.

In certain embodiments, a control feedback loop is provided. For example, during or following a particular electric energy applying protocol, a biological aspect of a subject may be monitored, e.g., by sensing conduction in a neuronal system, e.g., in a particular electrically stimulated nerve fiber, or by any suitable method. For example, a sensor suitable for detecting nerve cell or axon activity may be implanted in a portion of a subject's body. A sensor may take the form of an electrode or the like. Signals received by such a sensor may be amplified before further processing. A sensor may also take the form of a device capable of detecting nerve compound action potentials or may take the form of a transducer that includes an electrode with an ion selective coating applied which is capable of directly transducing the amount of a particular transmitter substance or its breakdown by-products. In utilizing a feedback system, if a predetermined detection criteria is not detected the same or a different stimulus protocol may be performed and may be automatically initiated under the control of a controller. For example, in those instances where a different protocol is performed, one or more of the electrical energy applying parameters may be modified, e.g., the pulse width may be increased, or the like, in the second protocol.

Modulating a Receptor to at Least Maintain Plasticity

Embodiments include modulating a receptor to lower LTP in a manner effective to at least maintain, e.g., increase, plasticity in a system of a subject. A variety of different receptors may be modulated and include, but are not limited to, melatonin receptors, melanocorticotropin receptors, melanocyte-stimulating receptors, bombesin receptors, galanin receptors, gastrin receptors, ghrelin receptors, gonadotropin releasing hormone receptors, follicle stimulating hormone receptors, leutinizing hormone receptors, growth hormone receptors, somatomedin receptors, somatostatin receptors, oxytocin receptors, progesterone receptors, relaxin receptors, serotonin receptors, and vasopressin receptors. Receptors may be modulated by applying electrical energy to the subject and/or by administering a pharmacological agent to the subject.

In modulating a receptor such as a receptor described above, embodiments may include administering an effective amount of one or more of the following: bombesin antagonists, galanin antagonists, gastrin agonists, melatonin agonists, melatonin antagonists, melanocorticotropin agonists, melanocorticotropin antagonists, melanocyte-stimulating hormone agonists, melanocyte-stimulating hormone antagonists, gastrin antagonists, ghrelin agonists, ghrelin antagonists, gonadotropin releasing hormone agonists, gonadotropin releasing hormone antagonists, follicle stimulating hormone agonist, follicle stimulating hormone antagonists, leutinizing hormone agonists, leutinizing hormone antagonists, growth hormone agonists, growth hormone antagonists, somatomedin agonists, somatomedin antagonists, oxytocin agonists, oxytocin antagonists, progesterone agonists, progesterone antagonists, relaxin agonists, serotonin agonists, serotonin antagonists, vasopressin agonists, vasopressin antagonists.

Modulating a Biological Pathway to at Least Maintain Plasticity

Embodiments also include modulating a biological pathway, e.g, a synthesis pathway, of a hormone or neuropeptide affecting any of the above described receptors to at least maintain, e.g., increase, plasticity in a system of a subject, where modulation includes up-regulating or down-regulating a pathway such as up- or down-regulating a pathway in the production of a hormone or neuropeptide in a subject. For example, embodiments include modulation of a melatonin synthesis pathway, e.g., increasing production of melatonin, melanocorticotropin, melanocyte stimulating hormone, tryptophan, serotonin, etc. Embodiments also include modulation of a pathway of any one of: bombesin, galanin, gastrin, ghrelin, gonadotropin releasing hormone, follicle stimulating hormone, leutinizing hormone, growth hormone, somatomedin, somatostatin, oxytocin, progesterone, relaxin, serotonin, and vasopressin.

Utility

The subject methods find use in a variety of applications in which it is desired to treat plasticity in a subject. By “treatment” is meant that the plasticity in a system of a subject is at least maintained, where in certain embodiments the plasticity in a system of a subject is increased. For example, as described above, embodiments of the subject methods include determining the plasticity in a system of a subject and then lowering LTP in the subject in a manner to at least maintain the plasticity at the level determined prior to lowering LTP, or to increase the plasticity to a level greater than the level determined prior to lowering LTP.

A variety of subjects are treatable according to the subject methods. In many embodiments the subjects are “mammals” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects are humans. Human subject of both genders and at any stage of development (i.e., neonates, infant, juvenile, adolescent, adult), may be treated according to the present invention, where in certain embodiments the human subject is a juvenile, adolescent or adult. While the present invention may be used for the treatment of a human subject, it is to be understood that the subject methods may also be carried-out on other animal subjects such as, but not limited to, birds, mice, rats, dogs, cats, livestock and horses. Accordingly, it is to be understood that any subject in need of being treated according to the subject invention is suitable.

Computer Readable Mediums and Programming Stored Thereon

The subject invention includes computer readable media having programming stored thereon for implementing the subject methods. For example, the subject invention may include suitable computing means such as suitable hardware/software for performing the subject methods.

In certain embodiments, programming may control a device to administer a pharmacological agent to subject in a manner to lower LTP, e.g., programming may be configured to determine suitable dosage, etc. In certain embodiments programming may control a device to administer electrical energy to subject in a manner to lower LTP, e.g., may control the activation/termination of electrical energy including selecting suitable electrical parameters. Programming may be configured to, or otherwise be capable of, directing a microprocessor to activate, i.e., turn “on” and “off” an electric energy applying device for applying energy to lower LTP in a subject. For example, if it is determined that LTP needs to be lowered (which determination may be made by the processor or otherwise communicated to the processor), the processor may direct the electric energy applying device to provide the appropriate energy to result in the desired action. Accordingly, a processor may select the appropriate parameters (e.g., frequency, amplitude, etc.) depending on what is required and direct an electric energy applying device to implement the parameters.

Programming according to the subject invention may be recorded on computer-readable media, e.g., any medium that can be read and accessed directly or indirectly by a computer. Such media include, but are not limited to, computer disk or CD, a floppy disc, a magnetic “hard card”, a server, magnetic tape, optical storage such as CD-ROM and DVD, electrical storage media such as RAM and ROM, and the hybrids of these categories such as magnetic/optical storage media. One of skill in the art can readily appreciate how any of the presently known computer readable mediums may be used to provide a manufacture that includes a recording of the present programming/algorithm for carrying out the above-described methodology. Thus, the computer readable media may be, for example, in the form of any of the above-described media or any other computer readable media capable of containing programming, stored electronically, magnetically, optically or by other means. As such, stored programming embodying steps for carrying-out some or all of the subject methods may be transferred to a computer-operated apparatus such as a personal computer (PC) or the like, by physical transfer of a CD, floppy disk, or like medium, or may be transferred using a computer network, server, or other interface connection, e.g., the Internet.

For example, the subject invention may include a computer readable medium that includes stored programming embodying an algorithm for carrying out the subject methods, where such an algorithm is used to direct a processor or series of processors to execute the steps necessary to perform the task(s) required of it and as such in certain embodiments the subject invention includes a computer-based system for carrying-out some or all of the subject methods. For example, such a stored algorithm may be configured to, or otherwise be capable of, directing a microprocessor to receive information directly or indirectly from data gathering means (i.e., information collected by data gathering means about LTP) and process that information to determine LTP in a subject and even whether LTP requires lowering, e.g., to achieve a certain increase in plasticity. The result of that processing may be communicated to a user, e.g., via audio and/or visual means, e.g., the algorithm may also include steps or functions for generating a variety of LTP profile graphs, plots, etc.

The subject invention may also include a data set of known or reference information stored on a computer readable medium to which LTP data collected may be compared for use in determining LTP and, e.g., whether LTP is in need of lowering. The data may be stored or configured in a variety of arrangements known to those of skill in the art.

Kits

Also provided are kits for practicing the subject methods. Kits may include an electric energy applying device, as described above. Devices for delivering, e.g., implanting, an electric energy applying device to a target site of a subject such as into the body cavity of a subject may also be included in the subject kits. For example, an endoscope, introducer needle, and the like may be provided.

The subject kits may also include one or more pharmacological agents for lowering LTP in a subject, as described above. The dosage amount of the one or more pharmacological agents provided in a kit may be sufficient for a single application or for multiple applications. Accordingly, in certain embodiments of the subject kits a single dosage amount of a pharmacological agent is present and in certain other embodiments multiple dosage amounts of a pharmacological agent may be present in a kit. In those embodiments having multiple dosage amounts of pharmacological agent, such may be packaged in a single container, e.g., a single tube, bottle, vial, and the like, or one or more dosage amounts may be individually packaged such that certain kits may have more than one container of a pharmacological agent.

Suitable means for delivering one or more pharmacological agents to a subject may also be provided in a subject kit. The particular delivery means provided in a kit is dictated by the particular pharmacological agent employed, as describe above, e.g., the particular form of the agent such as whether the pharmacological agent is formulated into preparations in solid, semi-solid, liquid or gaseous forms, such as tablets, capsules, powders, granules, ointments, solutions, suppositories, injections, inhalants and aerosols, and the like, and the particular mode of administration of the agent, e.g., whether oral, buccal, rectal, parenteral, intraperiactivityal, intradermal, transdermal, intracheal, etc. Accordingly, certain systems may include a suppository applicator, syringe, I.V. bag and tubing, electrode, etc.

The subject kits may also include instructions for how to practice the subject methods using the components of the kit, e.g., how to use an electric energy applying device if provided in the kit to lower LTP in a subject or how to administer a pharmacological agent to a subject if provided in the kit. The instructions may be recorded on a suitable recording medium or substrate. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Some or all components of the subject kits may be packaged in suitable packaging to maintain sterility. In many embodiments of the subject kits, the components of the kit are packaged in a kit containment element to make a single, easily handled unit, where the kit containment element, e.g., box or analogous structure, may or may not be an airtight container, e.g., to further preserve the sterility of some or all of the components of the kit.

### EXPERIMENTAL

The following examples are put forth so as to provide those of ordinary skill in the art with a complete disclosure and description of how to make and use the present invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Use of Melatonin to Decrease LTP and Increase Song Plasticity in Songbirds

Double blinded randomized controlled studies are used to ascertain the effect of melatonin on plasticity in songbirds. Methods of song plasticity evaluation are performed as described in Williams et al., but with substitution of melatonin administration for testosterone and placebo for flutamide in the study groups (Williams H, Connor D M, Hill J W. Testosterone decreases the potential for song plasticity in adult male zebra finches. Horm. Behav. 2003; 44(5):402-12.). LTP is concurrently measured through the treatment period. It is observed that melatonin administration with a concomitant decrease in LTP produces a statistically significant improvement in songbird plasticity as compared to control, and that this difference increases over time.

### Example 2

### Use of Melatonin to Decrease LTP and Increase Cognitive Plasticity in Humans

Double blinded randomized controlled studies are employed to ascertain the effect of melatonin administration on cognitive plasticity. Subjects in a treatment group are given melatonin with the intent of lowering LTP. A group of appropriate age-matched controls receive placebo. Each group performs a set of standardized learning tasks at predetermined time points in conjunction with measurement of LTP. Such tasks include, but are not limited to, language and music. Melatonin administration with a concomitant decrease in LTP is observed to produce a statistically significant improvement in learning ability and/or a statistically significant decrease in learning decrement for the treatment group as compared to control, and this difference increases over time.

### Example 3

### Use of Serotonin to Decrease LTP and Increase Scarring Plasticity in Humans

Double blinded randomized controlled studies are employed to ascertain the effect of serotonin administration on scarring plasticity. Subjects in a treatment group are given serotonin with the intent of lowering LTP. A group of appropriate age-matched controls receive placebo. Each group undergoes controlled wounding at identical sites on the body at predetermined time points in conjunction with measurement of LTP. Quantitative measurement of wound healing time and extent/thickness of scar is performed on a more frequent basis following each incident of wounding. It is observed that serotonin administration with a concomitant decrease in LTP produces a statistically significant improvement in wound healing time and/or a statistically significant decrease in scar extent/thickness for the treatment group as compared to control, and that this difference between the two groups increases over time.

### Example 4

### Neuromodulation to Decrease LTP and Increase Allergy Plasticity in Humans

Double blinded randomized controlled studies are employed to ascertain the effect of neuromodulation on allergy plasticity. Subjects in a treatment group have neuromodulation devices implanted and appropriate nerves/regions of the brain stimulated with electrical energy with the intent of lowering LTP. A group of appropriate age-matched controls have devices implanted but undergo no such stimulation. Challenge with a predetermined set of skin test allergens and measurement of LTP is performed at predetermined time points. It is observed that neuromodulation with a concomitant decrease in LTP produces a statistically significant decrease in number and extent of positive allergic responses to challenge for the treatment group as compared to control, and that this difference between the two groups increases over time.

All publications and patents and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of at least maintaining plasticity in a system of a subject, said method comprising:
lowering long term potentiation (LTP) in said subject in manner effective to at least maintain plasticity of a system in said subject.

2. The method of claim 1, wherein said system is said subject's neuronal system.

3. The method of claim 2, wherein said plasticity is cognitive plasticity.

4. The method of claim 1, wherein said system is said subject's immune system.

5. The method of claim 4, wherein said plasticity is allergy plasticity.

6. The method of claim 1, wherein said system is said subject musculoskeletal system.

7. The method of claim 6, wherein said plasticity is cartilage plasticity.

8. The method of claim 1, wherein said system is a wound healing system of said subject.

9. The method of claim 8, wherein said plasticity is scarring plasticity.

10. The method of claim 1, wherein said lowering comprises administering an effective amount of at least one of: melatonin, a melatonin analogue, a melatonin precursor, bombesin antagonists, galanin antagonists, gastrin agonists, melatonin agonists, melatonin antagonists, melanocorticotropin agonists, melanocorticotropin antagonists, melanocyte-stimulating hormone agonists, melanocyte-stimulating hormone antagonists, gastrin antagonists, ghrelin agonists, ghrelin antagonists, gonadotropin releasing hormone agonists, gonadotropin releasing hormone antagonists, follicle stimulating hormone agonist, follicle stimulating hormone antagonists, leutinizing hormone agonists, leutinizing hormone antagonists, growth hormone agonists, growth hormone antagonists, somatomedin agonists, somatomedin antagonists, oxytocin agonists, oxytocin antagonists, progesterone agonists, progesterone antagonists, relaxin agonists, serotonin agonists, serotonin antagonists, vasopressin agonists, vasopressin antagonists.

11. The method of claim 10, wherein said method comprises administering a melatonin precursor and said precursor is serotonin or a tryptophan or analogue of serotonin or tryptophan.

12. The method of claim 1, wherein said lowering comprises modulating a receptor.

13. The method of claim 12, wherein said receptor is selected from the group of: melatonin receptors, melanocorticotropin receptors, and melanocyte-stimulating hormone receptors.

14. The method of claim 12, wherein said receptor is a neuropeptide receptor.

15. The method of claim 14, wherein said neuropeptide receptor is selected from the group of: bombesin receptors, galanin receptors, gastrin receptors, ghrelin receptors, gonadotropin releasing hormone receptors, follicle stimulating hormone receptors, leutinizing hormone receptors, growth hormone receptors, somatomedin receptors, somatostatin receptors, oxytocin receptors, progesterone receptors, relaxin receptors, serotonin receptors, and vasopressin receptors.

16. The method of claim 1, wherein said lowering comprises modulating a biological pathway.

17. The method of claim 16, wherein said biological pathway is a melatonin pathway.

18. The method of claim 16, wherein said biological pathway is selected from the group of: a bombesin pathway, galanin pathway, gastrin pathway, ghrelin pathway, gonadotropin releasing hormone pathway, follicle stimulating hormone pathway, leutinizing hormone pathway, growth hormone pathway, somatomedin pathway, somatostatin pathway, oxytocin pathway, progesterone pathway, relaxin pathway, serotonin pathway, and vasopressin pathway.

19. The method of claim 1, wherein said method is a method of increasing plasticity of a system in said subject.

20. The method of claim 1, wherein said method further comprises, prior to lowering LTP, determining said plasticity.

21. An algorithm recorded on a computer-readable medium for administering at least one of a pharmacological agent and electrical energy to said subject in accordance with method of claim 1.

22. A system comprising:
(a) an algorithm recorded on a computer-readable medium for administering a pharmacological agent to a subject according to a method of claim 1;
(b) an effective amount of a pharmacological agent, and
(c) a drug delivery device.

23. A system comprising:
(a) an algorithm recorded on a computer-readable medium for administering electrical energy to a subject according to a method of claim 1;
(b) an electrical energy applying device.

24. A kit for at least maintaining plasticity in a system of a subject, said kit comprises:
a pharmacological agent; and
instructions for administering said pharmacological agent to a subject according to the method of claim 1.

25. A kit for at least maintaining plasticity in a system of a subject, said kit comprises:
an electric energy applying device; and
instructions for using said electric energy applying device according to the method of claim 1.
